(19) United States
(12) Patent Application Publication
(10) Pub. No.: US 2021/0162078 A1
(43) Pub. Date: Jun. 3, 2021

(54) METHOD FOR DIAGNOSIS OF DOPAMINERGIC AND MOVEMENT DISORDERS

(71) Applicant: LikeMinds, Inc., Boston, MA (US)

(72) Inventors: Kenneth L. RICE, Boston, MA (US); Peter G. SAVAS, Boston, MA (US)

(21) Appl. No.: 17/265,850

(22) PCT Filed: Aug. 7, 2019
(86) PCT No.: PCT/US2019/045472

§ 371 Date: Feb. 4, 2021

Related U.S. Application Data
(60) Provisional application No. 62/741,031, filed on Oct. 4, 2018.
(60) Provisional application No. 62/765,007, filed on Aug. 17, 2018.
(60) Provisional application No. 62/715,338, filed on Aug. 7, 2018.

(51) Int. Cl. A61K 51/04

(52) U.S. Cl. CPC A61K 51/0448

(57) ABSTRACT

This disclosure relates to methods of determining if a subject not manifesting any clinical symptoms of a dopaminergic disorder is afflicted with the dopaminergic disorder using radiolabeled DatT020 or a derivative thereof.

### PRIORITY CLAIM

This application claims priority to U.S. Provisional application No. 62/715,338 filed Aug. 7, 2018; 62/765,007, filed Aug. 17, 2018; and 62/741,031, filed Oct. 4, 2018, the contents of which are hereby incorporated herein in their entirety.

### FIELD OF THE INVENTION

The present invention is in the field of human and veterinary medicine. More specifically, the invention relates to methods of diagnosis of dopaminergic and non-dopaminergic disorders.

### BACKGROUND OF THE INVENTION

Movement disorders are common among adults and have significant personal, financial, and societal impact. Varying degrees of tremor are often manifested, with severe tremor causing difficulty or the inability to perform routine activities. Many of these disorders are progressive and may proceed rapidly (Zesiewicz et al. (2005) Neurol. 64(12): 2008-20; Stone (1991) Pharmacol. Biochem. Behav. 39(2): 345-9; Stone (1995) J. Neurol. Sci. 132(2):129-32.

Movement disorders are usually or initially diagnosed clinically and may be based on questionnaires, non-motor symptoms such as REM sleep disorders, constipation, and observation of voluntary and involuntary movement. For example, in the case of the most common movement disorder, non-parkinsonian tremor (also called Essential tremor, benign tremor, familial tremor, or idiopathic tremor), diagnosis is usually established by observing an action tremor (i.e., a tremor which intensifies when one tries to use the affected muscles of hands, arms, and/or fingers) or postural tremor (i.e., present with sustained muscle tone), rather than the tremor exhibited at rest (resting tremor), which is manifested in Parkinson's Disease. When a limb is at rest, no tremor is observed, but moving or extending the limb results in a shaking. This is in contrast to resting tremor which is manifested in Parkinson's disease.

Unfortunately, clinical diagnoses of movement disorders are at best speculative many disorders display similar physical symptoms which can also be confused with other tremor disorders.

Definitive diagnosis of a movement disorder is possible only during autopsy when histological examination of regions of the brain, e.g., the basal ganglia, particularly the substantia nigra (SN), is performed. This is because the dopaminergic pathway from the SN to the striatum is known to play an integral part in motor function and is involved in many movement disorders (“dopaminergic” disorders). However, not all tremor disorders involve this pathway. For example, there are movement disorders that, in the earlier stages, may manifest as Parkinson's Disease clinically, but there is no evidence of dopamine deficiency in the striatum, thus suggesting the movement disorder etiology is a type of non-parkinsonian syndrome (such as essential tremor, a “non-dopaminergic movement disorder) which requires a different clinical treatment protocol. Therefore, without information to rule out the involvement of the SN to striatum dopamine pathway, clinical diagnoses of movement disorders are at best speculative.

More recently, the SN to striatum pathway of a live patient has become assessible by viewing images of the striatum obtained by various noninvasive imaging techniques. Such non-invasive methods use single photon emission computed tomography (SPECT) assessment measuring the uptake/binding of a radiolabeled imagining agent that binds to dopamine transporters (DAT) in the striatum. Currently, the only SPECT radiopharmaceutical approved for use in the United States, the European Union, and Canada is DaTscan® (N-ω-fluoropropyl-2β-carbomethoxy-3β-(4-iodophenyl) nortropane), which is radiolabeled with ¹²³I. After injection of DaTscan, the patient must wait 3 hr for its uptake into systemic circulation, and transition through the blood brain barrier into the parenchyma of the brain tissue until it reaches stable binding to DAT. At that point, radioactivity (counts) can be collected and quantified, and a visual image can be acquired via SPECT. The visual image is compiled for reading by experts who then determine if there is evidence of DAT density loss in the striatum. In the case of a patient with Parkinson's disease, an asymmetrical pattern of binding in the striatum is observed in the image, whereas in the non-parkinsonian tremor patient, the pattern is visible as bilateral and symmetrical (i.e., “normal) (Lewis et al. (2012) Practitioner 1748: 21-24). This information is used by treating physicians in determining diagnosis and subsequent treatment.

Unfortunately, DaTscan is also highly selective for serotonin (SERT) transporters (for every two to three DAT sites DaTscan binds to one SERT site) in the lungs and other tissues (DaTscan™ Ioflupane ¹²³I Injection (package insert), Arlington Heights, Ill.: GE Healthcare, Medi-Physics, Inc.; 2015). Due to DaTscan's selectivity for SERT and the slow transition time of the compound to the neurons in the SN, imaging cannot take place until 3 to 6 hr after injection, at which time its binding to striatal DAT becomes stable. The patient is then placed in the SPECT camera until 1.5 million counts are obtained (approximately 45 min). The binding of DaTscan to transporters other than DAT leaves only about 7% of DaTscan available for DAT transporter binding in the brain tissue (DaTscan™ Ioflupane ¹²³I Injection (package insert), ibid). Thus, a significant disadvantage of the only currently approved imaging agent used with this technology is that an undetermined but significant amount of DaTscan is being bound and captured by serotonin transporters throughout the body and, therefore, is not available for specific binding to neuronal DAT in the striatum.

Therefore, what is needed is a more accurate and faster way to diagnose movement disorders which can distinguish non-dopaminergic disorders dopaminergic disorders so that efficacious therapeutic treatment specific for the disorder can be administered more quickly.

DAT is also found outside the brain in nephrons, kidney, pancreas, lungs, and the cardio-pulmonary system including many blood vessels outside the CNS. Dysfunctions of DAT outside the brain are known. Thus, what is also needed are ways to diagnose DAT related disorders outside of the brain.

### SUMMARY OF THE INVENTION

It has been discovered that the imaging agent [¹²³I]-E-2β-carbomethoxy-3β-(4-fluorophenyl)-N-(3-iodo-E-allyl) nortropane (DaT2020) has a binding selectivity of 28-fold for DAT over SERT. This enables an increase in the availability of tracer which can penetrate more deeply into brain tissues, and can bind more quickly to DAT than other known radiolabeled tropane imaging agents.

These discoveries have been exploited to provide the present methods useful to quickly and accurately determine if a patient afflicted with a movement disorder has a malfunction of the dopaminergic system or has a non-dopaminergic movement disorder or other disorder that is difficult to diagnose through clinical observations alone.

In one aspect, the present disclosure describes methods of determining if a patient manifesting active tremor symptoms is afflicted with a non-dopaminergic or dopaminergic movement disorder. This method comprise administering radiolabeled DaT2020, or a radiolabeled derivative thereof, to the patient; acquiring counts from the radiolabeled DaT2020, or derivative thereof, bound to DAT in the striatum of the patient, with initiation of the acquisition of counts beginning at about 15 min after administration; measuring a number, pattern, or density of counts acquired; and comparing the number, density, and/or pattern of counts acquired from the striatum of the patient with the number, density, and/or pattern of counts obtained from an unafflicted, age-matched control subject. If the patient is afflicted with a non-dopaminergic movement disorder, the number or density of counts detected in the striatum of the patient is about the same or similar relative to the counts obtained from an unafflicted subject. If the patient is afflicted with a dopaminergic movement disorder, the counts detected from the patient are reduced relative to the counts obtained from an unafflicted (normal) subject.

In some embodiments, DaT2020, or a derivative thereof, is radiolabeled with ¹²³I, ¹²⁴I, ¹²⁵I, ^{99m}Tc, ¹⁸F or ^{117m}Sn. In particular embodiments, DaT2020, or a derivative thereof, is radiolabeled with ¹²³I, ¹²⁵I, ^{99m}Tc, or ^{117m}Sn, and counts are acquired by SPECT. In other embodiments, DaT2020, or a derivative thereof, is radiolabeled with ¹⁸F, ¹²⁴I, or ¹¹C, and the counts are acquired by PET.

In some embodiments, about 1 mCi to about 10 mCi ¹²³I-labeled DaT2020, or a derivative thereof, is administered to the patient. In other embodiments, about 3 mCi to about 5 mCi ¹²³I-labeled DaT2020, or a derivative thereof, is administered to the patient.

In certain embodiments, the derivatives of DaT2020 comprise 2β-carbomethoxy-3β-(4-iodophenyl) tropane beta-CIT); 2β-carbomethoxy-3β-(4-iodophenyl)-N-(3-fluoropropyl)nortropane (FP-CIT) and TRODAT-1.

In some embodiments, the non-dopaminergic disorder afflicting the patient is essential tremor. In other embodiments, the dopaminergic disorder afflicting the patient is Parkinson's disease, Lewy Body dementia, or diabetes.

In yet other embodiments, the counts are acquired for at least 30 min, and the method further comprises compiling an image of DAT bound to radiolabeled DaT2020, or a derivative thereof, in the striatum of the patient, the image being symmetrical if the patient is afflicted with a non-dopaminergic movement disorder, and the image being asymmetrical if the patient is afflicted with a dopaminergic disorder. In some embodiments, the image is compiled from counts acquired by PET and in other embodiments, the image is compiled from counts acquired by SPECT.

In another aspect, the disclosure provides s method of determining if a subject not manifesting a clinical symptom of a dopaminergic disorder is afflicted with that dopaminergic disorder. The method comprises: administering radiolabeled DaT2020, or a radiolabeled derivative thereof, to the subject; acquiring counts from the radiolabeled DaT2020, or derivative thereof, bound to DAT in a region of interest (ROI) of the body of the subject, initiation of the acquisition of counts beginning at about 15 min after administration; measuring a number, density, and/or pattern of counts acquired; and comparing the number, density, and/or pattern of counts acquired from the ROI of the subject with the number, density, and/or pattern of counts obtained from an unafflicted, age-matched control subject. If the patient is afflicted with a dopaminergic movement disorder, the number, density and/or pattern of counts detected in the ROI is reduced relative to the counts, density, and/or pattern of counts obtained from the ROI the unafflicted, age-match control subject.

In some embodiments, the method further comprises repeating the method at a set period(s) of time after the method is first performed.

In certain embodiments, the number, density, and/or pattern of counts obtained from the unafflicted, age-matched control subject s an average of counts, density, and/or patterns obtained from a plurality of unafflicted, age-matched control subjects.

In some embodiments, DaT2020, or a derivative thereof, is radiolabeled with ¹²³I, ¹²⁴I, ¹²⁵I, ^{99m}Tc, ¹⁸F or ^{117m}Sn. In particular embodiments, DaT2020, or a derivative thereof, is radiolabeled with ¹²³I, ¹²⁵I, ^{99m}Tc, or ^{117m}Sn, and counts are acquired by SPECT. In other embodiments, DaT2020, or a derivative thereof, is radiolabeled with ¹⁸F, ¹²⁴I, or ¹¹C, and the counts are acquired by PET.

In some embodiments, about 1 mCi to about 10 mCi ¹²³I-labeled DaT2020, or a derivative thereof, is administered to the patient. In other embodiments, about 3 mCi to about 5 mCi ¹²³I-labeled DaT2020, or a derivative thereof, is administered to the patient.

In certain embodiments, the derivatives of DaT2020 comprise 2β-carbomethoxy-3β-(4-iodophenyl) tropane beta-CIT); 2β-carbomethoxy-3β-(4-iodophenyl)-N-(3-fluoropropyl)nortropane (FP-CIT) and TRODAT-1.

In some embodiments, the dopaminergic disorder is Parkinson's disease or Lewy Body Dimentia.

### DESCRIPTION OF THE DRAWINGS

The foregoing and other objects of the present disclosure, the various features thereof, as well as the disclosure itself may be more fully understood from the following description, when read together with the accompanying drawings, in which:

FIG. 1 is a chemical representation of Dat2020;

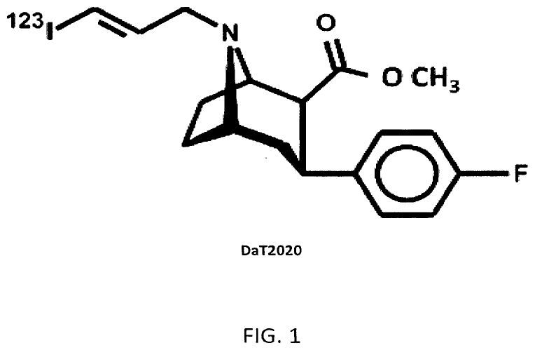

FIG. 2 is a diagrammatic representation summarizing DaTsnap processes; and

FIG. 3 is a graphic representation of the striatal binding ratios (ROI=striatum/surrounding non-striatal tissue) for DaT2020 (study 1) and for DaTScan (study 2) over a 60 min period after administration.

### DESCRIPTION

The disclosures of any patents, patent applications, and publications referred to herein are hereby incorporated by reference in their entireties into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, “DaT2020” refers to E-2β-carbomethoxy-3β-(4-fluorophenyl)-N-(3-iodo-E-allyl) nortropane.

The terms “agent” and “tracer” encompass radiolabeled DaT2020, and radiolabeled derivatives thereof.

The term “tropane” as used herein refers to DaT2020 and its derivatives.

The terms “radiolabeled tropane,” “radiolabeled DaT2020,” “tracer”, and “agent” as used herein refer to radiolabeled-DaT2020, and derivatives thereof, labeled with ¹²³I, ¹²⁴I, ¹²⁵I, 18F, ^{99m}Tc, ¹¹C, or ^{117m}Sn. “Altropane” refers specifically to [¹²³I E-2β-carbomethoxy-3β-(4-fluorophenyl)-N-(3-iodo-E-allyl) nortropane.

The present disclosure provides, at least in part, diagnostic methods using the radiolabeled imaging agent, DaT2020, or derivatives thereof, to quickly distinguish dopaminergic disorders from non-dopaminergic disorders, and to image dopamine transporters (DATs) in different regions of the brain and body involved in such disorders. This imaging agent is highly advantageous as it is more selective for, and binds more quickly to, DAT than other commercially available imaging agents.

### A. Conditions to be Assessed

The present diagnostic and imaging methods aid in the differential diagnosis, leading to appropriate treatment of conditions where the functioning or dysfunctioning of DAT is a biomarker. These methods can also be used in clinical trials designed to evaluate the efficacy of new treatments for DAT dysfunction to stratify subjects according to disease stage. These methods are also useful for monitoring the effectiveness of treatments for and progression of DAT dysfunction over time.

Dysfunctions of DAT resulting in dopaminergic disorders are known in the brain and CNS, as well as outside of the CNS, including pancreas, kidney, and cardiovascular system.

The present method can distinguish non-dopaminergic conditions, such as, but not limited to, non-parkinsonian or essential tremor and non-Alzheimer dementia, as well as multiple sclerosis, chronic kidney disease, stroke, traumatic brain injury, drug or alcohol use, hypoglycemia, lack of sleep, lack of vitamins, increased stress, magnesium and/or thiamine deficiencies, liver failure, mercury poisoning, and drug or alcohol addiction or withdrawal, from a dopaminergic disorder displaying similar clinical manifestations. Such dopaminergic disorders include, but are not limited to, parkinsonian syndromes including idiopathic Parkinson's disease, progressive supranuclear palsy (PSP), multiple system atrophy (MSA), corticobasal degeneration (CBD), and vascular parkinsonism (VaP), among other rarer causes of parkinsonism), and Lewy body dementia, ADHD, clinical depression, anxiety, sleep disorders, obesity, sexual dysfunction, schizophrenia, pheochromocytoma, binge eating disorder, and diabetes and other disorders resulting from DAT dysfunction outside of the CNS.

### B. DaT2020 and Derivatives

The imaging agent used in the present methods is the tropane, DaT2020, and derivatives thereof (collectively, “DaT2020”). These imaging agents have a higher binding selectivity for DAT over SERT (28:1 or 28-fold), bind more DAT quickly, and penetrate brain tissue and other regions of interest more deeply than other known DAT tropane tracers currently used (e.g., DaTscan) (DaTscan™ Ioflupane ¹²³I Injection [package insert] (2015) Arlington Heights, Ill.: GE Healthcare, Medi-Physics, Inc.).

Non-limiting examples of DaT2020 derivatives include 2β carbomethoxy-3β-(4-iodophenyl) tropane (beta-CIT); 2β-carbomethoxy-3β-(4-iodophenyl)-N-(3-fluoropropyl) nortropane (FP-CIT); and TRODAT-1. These derivatives are described in U.S. Pat. Nos. 5,493,026, 8,084,018, 8,574,545, 8,986,653, and PCT International Application No. PCT/US2015/037340. DaT2020 can be commercially obtained (from LikeMinds, Inc.) or can be synthesized, e.g., according to U.S. Pat. Nos. 8,986,653 and 8,574,545).

Radiolabeled DaT2020 and its radiolabeled derivatives may be generated by the user through a radiolabeling procedure. For example, to prepare DaT2020, one may allow a reaction between a haloallyl Sn precursor (pre-DaT2020) and a radionuclide under oxidative conditions. Other standard methods of radiolabeling can be used as well. For radiolabeling, DaT2020 in lyophilized form is useful, however, it can also be in aqueous form.

The radiolabel bound to the tropane is one that is detectable via SPECT (as shown above), including ¹²³I, ¹²⁴I, ¹²⁵I, ^{99m}Tc, or ^{117m}Sn or PET (as shown above), including ¹⁸F or ¹¹C. The location of the radioisotope on the agent can be varied. For example, the isotope can be located at any position on pre-DaT2020 or a derivative thereof and can be directly linked or indirectly linked via a linker (see, U.S. Pat. No. 8,574,545). One suitable position is the free terminus of the haloallyl moiety.

Non-limiting examples of useful SPECT-readable tracers for DAT detection according to the disclosure include [¹²³I]-E-2β-carbomethoxy-3β-(4-fluorophenyl)-N-(3-iodo-E-allyl) nortropane (DaT2020), [¹²³I]-2β carbomethoxy-3β-(4-iodophenyl)tropane ([¹²³I]-beta-CIT); [¹²³I]-2β-carbomethoxy-3β-(4-iodophenyl)-N-(3-fluoropropyl)nortropane ([¹²³I]-FP-CIT); [¹²³I]-altropane; and [⁹⁹mTc]-TRODAT-1. Among these, [¹²³I]-FP-CIT (DaTscan) achieves stable binding 3 hr post-injection and remains stable for 3 hr, has a half-life of 13.2 hr, emits gamma rays with an energy of 159 keV, and is FDA approved. These are also described in U.S. Pat. Nos. 5,493,026, 8,084,018, 8,574,545, 8,986,653, and PCT International Application No. PCT/US2015/037340. The movement, and binding, of radiolabeled DaT2020 to DAT can also be performed using other methods of monitoring radioactive compounds used, e.g., by radioactivity sensors located on the head of the patient adjacent to the region of interest (ROI), or on other regions of the body where DATs are located and are being monitored for DaT2020 binding.

### C. Pretreatment

In some cases, prevention of thyroid uptake of the iodine isotope is warranted. This can be accomplished by orally administering to the subject a Lugol solution, potassium iodide solution, or potassium perchlorate solution. In some cases, other preliminary steps are performed before the administration of radiolabeled DaT2020. For example, for SPECT detection and imaging of DAT binding, steps that counter serotonin-reuptake inhibitors, amphetamines, and sympathomimetics can be employed. The discontinuance of any medications that might interfere with the binding of DaT2020 to DAT may also be required. Such potentially interfering molecules include selective serotonin reuptake inhibitors and CNS stimulants. The thyroid blocker is administered per label instructions to ensure effective blocking before the scheduled administration of the agent.

### D. Preparation and Administering of Agent

To determine if a patient is afflicted with a non-dopaminergic or dopaminergic disorder, the patient is injected with a diagnostically effective amount of radiolabeled DaT2020.

The radiolabeled DaT2020 can be formulated for intravenous (IV) systemic or direct local administration in a carrier or physiological buffer that does not inhibit its binding to DAT. Such buffers include, but are not limited to, ethanol (solvent), sodium hydroxide and acetic acid (pH adjustment), sodium chloride injection (isotonic vehicle). The formulation to be administered as a bolus injection has been shown in sponsored clinical trials to be safe containing a dose of up to about 8 mCi to 10 mCi of radiolabeled DaT2020 and delivered in a total volume of about 2.5 mL to about 5 mL.

The radioactivity of the dose of radiolabeled DaT2020 or of its radiolabeled derivatives, can be determined one with skill in the art, e.g. by a nuclear medicine imaging technician. In addition, the total radioactivity of the actual administered dose is of relevance and not the volume administered to achieve this dose. The exact radioactive dose of the tracer administered is determined by calculating the difference between the radioactivity in the syringe and delivery system before and after injection. After the dose is delivered, the syringe is filled with a volume of saline equal to the administered dose volume. The syringe content is recounted under the same conditions as used to determine the dose; separately. Useful dose ranges from about 1 mCi to about 10 mCi, from about 2 mCi to about 10 mCi, from about 3 mCi to about 7 mCi, from about 5 mCi to about 8 mCi, or about 2 mCi, about 3 mCi, about 4 mCi, about 5 mCi, about 6 mCi, about 7 mCi, about 8 mCi, about 9 mCi, or about 10 mCi. Dose can alternatively be described as effective dose (for 5 mCi of radiolabeled DaT2020) of approximately 4.3 mSv. Injected dose values outside the above stated range, i.e., values lower than about 1 mCi or higher than about 10 mCi are considered as potential sources of variation.

DaT2020 or derivatives formulated as described above for IV delivery are administered in a single (bolus) dose via a syringe (e.g., a peripheral 18 to 22 gauge venous catheter inserted for the radiopharmaceutical/tracer infusion). After injection, the tracer passes through the blood-brain barrier and quickly binds to DAT if it is available. Other methods of administration can also be utilized, for example, the direct injection of suitable amounts into the brain arteries via a syringe or catheters following established procedures in neuroradiology, or arteries contiguous to the striatum or other ROI.

### E. Count Acquisition

Before agent administration, the subject is positioned in a SPECT or PET camera. Alternatively, sensors are placed adjacent to the ROI on the body which can collect radioactivity and which interfaces with a reader that communicates data to a computer.

SPECT and PET are procedures in which the isotope bound to DaT2020 or derivatives thereof is measured once the agent has been administered and has reached stable binding in the ROI. Once entering the blood stream, it travels throughout the body (e.g., brain, liver, kidney, heart, lungs, and the peripheral vascular system) and binds to DAT in various regions of the body including the striatum. The time it takes to reach stable binding (to eliminate background noise to form a clearer image) depends on the ROI and the depth of the tissue penetrated by the agent.

The camera captures energy produced by the radioactive decay of the radiolabeled DaT2020 or derivatives thereof (“tracer”). The radiolabel used for SPECT imaging emits energy in the form of individual photons or gamma rays, while the radiolabel used for PET imaging emits energy in the form of positrons. These positrons almost immediately collide with an electron and the energy produced becomes 2 photons emitted at roughly 180 degrees (2 photons moving in opposite directions).

In SPECT, the single emitted photon passes into the camera and strikes a scintillation crystal which, in turn, is viewed by a large number of photomultiplier tubes. The output voltages generated by the photomultiplier tubes are fed to a position circuit which produces four output signals. These signals contain information about the position and intensity of where the photon struck the crystal (scintillations). These signals (which “unblank” or “light up” the receiver, e.g., a cathode ray oscilloscope) for each photon are then fed into the memory circuitry of a computer and stored. The storage of these signals (the position and intensity of each photon becomes a count) allows their recall for digital processing from distinct time points or periods as well as the distinguishing counts from the components that make up the ROI that was scanned. Therefore, one can obtain counts and process them for quantitation at any single moment or over varying periods of time during a single scan session. In order to construct an image, a scan session must collect millions of counts over time in order to produce enough detail for a human reader to evaluate.

In PET, the collector mechanism and scintillation crystal are able to obtain position and intensity information from both photons and feed the information into the memory circuitry of a computer for storage and for later retrieval and processing.

Collection of counts is initiated at about 10 to 15 min after administration of the agent and is carried out for about 1 min to about 15 min.

### F. SPECT Image Acquisition

To obtain an image, counts are collected beginning 15 min after administration of the radiolabeled agent and collection continues for 30 min (or 15-45 min after administration).

SPECT acquisition is performed on a SPECT/CT or stand-alone SPECT with at least two imaging heads fitted with collimators (parallel-beam and fan beam collimators with manufacturer specified (or measured according to NEMA standards) planar system resolution of <8 mm FWHM (in ‘air’ at 10 cm distance). Raw projection data (or counts) are acquired as described in Djang et al. (2013) Nuclear Med. Mol. Imaging 47(2):73-80), step-and-shoot mode with angle increments of 3° can be used. Alternatively, continuous rotation may be used. Full 360° coverage of the area surrounding the ROI (e.g., when ROI is the striatum, the “area surrounding the ROI” would be the head) is required (i.e., 180° for each head of a dual-head camera). The number of sec per position depends on the sensitivity of the system, e.g., 30 sec to 40 sec.

The photopeak of the camera is set at 159 keV±10% and a 128×128 matrix is used. Optimal images are obtained when matrix size and zoom factors give a pixel size of 3.5 mm to 4.5 mm. Slices are about one pixel thick.

### G. SPECT Image Processing

As described in Djang et al., 2012, ibid., review of projection data in cine mode and sinograms is performed for an initial determination of scan quality, patient motion, and artifacts. Motion correction algorithms may be used before reconstruction for minor movements, but rescanning is necessary if there is substantial head motion.

Iterative reconstruction (ordered subset expectation maximization [OSEM]) can be used, but filtered back-projection may be used. The reconstructed pixel size is 3.5 mm to 4.5 mm with slices one pixel thick.

Attenuation correction is done using an attenuation map measured from a simultaneously or sequentially acquired transmission or CT scan, or can be calculated, as with a correction matrix (see, Maebatake et al. (2015) J. Nucl. Med. Technol. 43: 41-46. doi:10.2967/jnmt.114.149401). The broad-beam attenuation coefficient is about 0.11 cm. Accuracy may be verified with an appropriate ¹²³I phantom (American College of Radiology (ACR). (2016). Site Scanning Instructions for the ACR Nuclear Medicine Phantom. In (pp. 18). Reston, Va.: American College of Radiology).

A low-pass filter (e.g., Butterworth) (Akahoshi et al. (2017) Medicine 96(45), e8484. doi:10.1097/md.0000000000008484) is useful. The filter preserves the linearity of the count rate response. Filtering includes either a 2-dimensional pre-filtering of the projection data or a 3-dimensional post-filtering of the reconstructed data.

Images are reformatted into slices in at least three planes depending on the ROI (axial, coronal, and sagittal). Transverse slices are parallel to a standard and reproducible anatomic orientation, such as the anterior commissure-posterior commissure line as used for brain MRI. This can be approximated by orientating the brain such that the inferior surface of the frontal lobe is level with the inferior surface of the occipital lobe. The canthomeatal plane, as routinely used for CT, is also acceptable. Activity in the striatum and the parotid glands, and the contours of the brain and the head, can usually be seen and can be used to assist realignment. A simultaneously acquired CT scan may allow precise realignment of the head.

Interobserver (expert image reader) variability is reduced by rigorously standardizing realignment and using predefined ROIs that are at least twice the full width at half maximum. Typically, this results in a smallest ROI dimension of 5 pixels to 7 pixels. In addition, three consecutive slices in the target region are used—those with the highest activity. Within the same center, the number of slices chosen are kept consistent.

### H. PET Image Acquisition

After tracer administration, (with a tracer radiolabeled for scanning by PET, e.g., F-18), ‘PET scans are acquired for 10 min with the patient's eyes open in a dimly lit room with minimal auditory stimulation. Imaging acquisition is performed using a high-resolution PET-CT scanner (Gemini TF, Philips Medical Systems, Cleveland, Ohio) from the skull vertex to the base. PET scanner generates 90 contiguous transverse slices with an intrinsic resolution of 4.4 mm full-width half-maximum (FWHM) in all directions and an axial field of view of 18 cm. Attenuation correction is performed using a low-dose CT scan, 16-slice multidetector helical CT unit using the following parameters: 120 kVp; 30 mA; 0.5-s rotation time; 1.5-mm slice collimation, 2-mm scan reconstruction, with a reconstruction index of 2 mm; 60-cm field of view; 512×512 matrix.

### I. PET Image Processing

Data acquired for imaging are reconstructed iteratively using a three-dimensional row action maximum-likelihood algorithm (RAMLA), often with low-dose CT datasets for attenuation correction in 3D mode.

Image processing and calculation can be performed using Statistical Parametric Mapping 2 software (SPM2, Wellcome Department of Imaging Neuroscience, University College of London, UK) in conjunction with MATLAB version 7.0 (MathWorks Inc., Natick, Mass.) and FIRE (Functional Image Registration, Seoul National University, Seoul, Korea) program [24]. Image datasets of CTI format can be converted to ANALYZE format using the software MRIcro (www.mricro.com, Rorden and Brett, Columbia, S.C.).

### J. Visual Evaluation of Image for DAT Density

The counts can be acquired until binding in the ROI is stable, and the method further comprises compiling an image of radiolabeled DaT2020, or a derivative thereof, bound to DAT in the ROI. For differential diagnosis of movement disorders, the ROI is the striatum of the patient. The image of the two halves is symmetrical if the patient is afflicted with a non-dopaminergic movement disorder, and the image is asymmetrical if the patient is afflicted with a dopaminergic disorder. The image is compiled from counts acquired by PET. Alternatively, the image is compiled from counts acquired by SPECT.

Alternatively, the pattern, level, and/or intensity of radiolabeled DaT2020 binding to DAT can be determined by the data captured by the sensors and sent, e.g., to a data reader attached to a computer. This method can be used for monitoring dopaminergic disorders affecting the brain or other ROI outside the brain.

### K. Quantitation

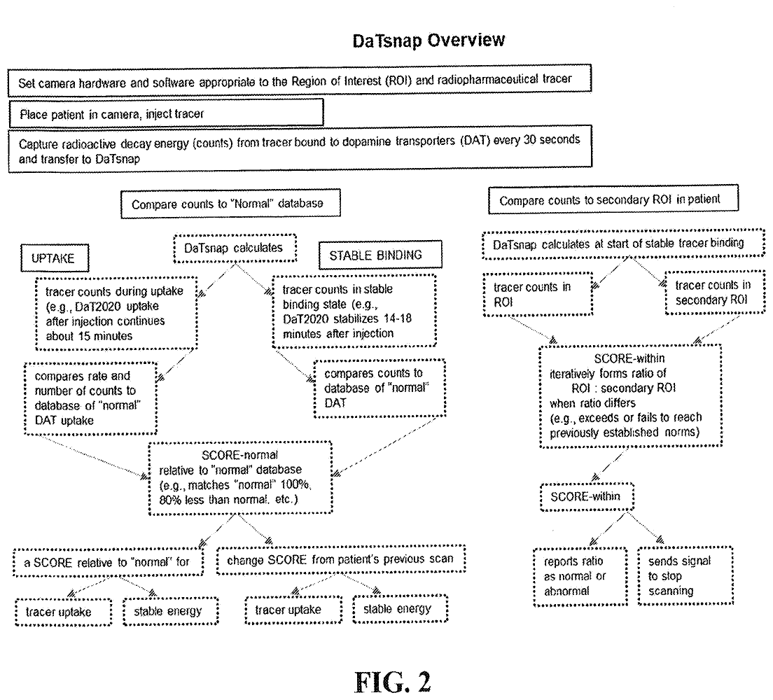

FIG. 2 depicts an example method for radiopharmaceutical tracer analysis for detection of a dopaminergic disorder, in an embodiment. The method depicted in FIG. 2 improves on prior analysis methods because it is performed significantly faster as it does not require a full image to be generated prior to analysis.

In the method shown in FIG. 2, the camera software and hardware is set appropriate to the ROI and radiopharmaceutical tracer. The patient is placed into the camera, and injected with the radiopharmaceutical tracer and scanning of the patient begins. When imaging striatal dopaminergic disorder, scanning of the patient must occur starting at about 15 min post-tracer administration to allow the tracer to achieve stable binding and continue for 30 min in order to construct an image. In contrast, the method of FIG. 2 begins scanning at the same time but only scans for 10 or 15 min and there is no necessity for constructing an image.

The camera scans the patient to capture energy in the form of counts (see Section E) from radioactive decay of the radiolabeled DaT2020 or derivatives thereof (“tracer”). This scan does not need to capture the total number of counts needed to construct a clear image of the ROI, “counts”. During the scan, counts are continuously collected by the camera at preset coordinates focusing on the organ of interest and then digitized, stored, and then processed in near real time. The scan continues until a predefined condition is met.

One example of the condition is a predetermined threshold reached. This threshold may be determined by the number or density of counts (a SCORE) that a person without a dopaminergic disorder would be expected to have as demonstrated by comparison to a database of counts from these persons (or to a striatal phantom). If this condition is met, (evidence there is normal DAT density) there is an indication that the patient does not have a dopaminergic disorder. Once this condition is met, the processor analyzing the counts may output a signal to stop the camera.

Another example of the condition is the determination that the amount of energy being captured has stabilized. This condition may be met when the number of counts quantified by the processor is similar over a predetermined time period (e.g., every 30 sec). In the case of radiolabeled DaT2020, the energy stabilizes typically between 14 to 18 min. If the energy stabilizes, and the above first condition (e.g., the number, threshold, range, of counts) is not met, then this is an indication that the patient may have a dopaminergic disorder. In such instance, the processor outputs a signal to continue scanning the patient in order to collect enough data to create an image. The captured data is then processed by computer algorithms (computed tomography) to create a visual image representation of the ROI showing bright white where the Tracer has bound to DAT against a dark background.

The signals output by the processor (e.g., to stop the camera or to continue scanning the patient in order to collect enough data to create an image) may be a signal automatically controlling operation of the camera, or it may be a signal (e.g., an audio, visual, or tactile signal) to the operator of the camera.

### L. Semi Quantification

Semiquantification is defined as the ratio of activity in a structure of interest (ROI) to activity in a reference region (Djang et al. (2012) ibid).

To date, the method used to limit human error in visually assessing images is to calculate a ratio of the counts from the ROI: counts from an area near the ROI that naturally has fewer dopamine transporters. It is this ratio [ROI-background/background] that provides a measure that is comparable regardless of tracer or camera used.

For example, if the goal is to determine if a movement disorder is parkinsonian (loss of DAT density) or non-parkinsonian the specific binding ratio (SBR, also referred to as the striatal binding ratio). SBRs are calculated after an image has been created by isolating (manually or through automation) the activity in the striatum and comparing it with activity in a low DAT density background area using the following formula:

$$Striatal binding ratio ( SBR ) = \frac{\begin{matrix}mean counts of straight ROI - \\ mean counts of background ROI\end{matrix}}{mean counts of background ROI}$$

For both manual and automated semiquantification, SBRs for the left and right striatum are quantified separately, and the caudate and putamen are quantified separately; known anatomic lesions may influence the location of the striatal or background ROIs.

Techniques roughly fall into four categories: classic manual ROIs, manual volumes of interest (VOIs), more advanced automated systems using VOIs, and voxel-based mathematic systems (DatQUANT (Ge Healthcare, Little Chalfont, UK)

The classic and most widely used method applies ROI templates manually to one or more slices with the highest striatal activity. Manual VOI strategies stress accurate characterization of the putamen as the most sensitive region for distinguishing normal findings from Parkinsonian syndromes. For sampling the putamen, a small VOI not encompassing the whole structure may be considered. Mid-putaminal VOIs provide accurate manual results. Automated VOI systems incorporating the whole striatum using individualized VOIs, either based on the ¹²³I-labeled tracer SPECT data or on a coregistered anatomic scan, produce more objective, observer-independent results and are faster although not widespread.

The following method improves on prior analysis methods because it is performed significantly faster as it does not require a full image to be generated prior to analysis. It also uses the patient as their own control. Software (DaTsnap) captures raw counts and calculates a caudate to putamen ratio (CPR) ratio of activity on the side of the brain contralateral to the movement disorder symptoms. The threshold for stopping the scan is when this ratio exceeds the normal ratio value.

$$CPR ( Caudate to putamen ratio ) = \frac{Mean count of caudate ROI}{Mean count of putamen ROI}$$

When this threshold is exceeded before the full imaging time has elapsed, the patient is afflicted with a dopaminergic movement disorder, the ratio of the patient's caudate to putamen will be higher relative to the normal ratio threshold. If the patient is afflicted with a non-dopaminergic movement disorder, the ratio of mean counts acquired from the patient's caudate to putamen will be the same, or similar to, the ratio obtained from an unafflicted subject.

To get an accurate determination of the level or amount of DAT in, e.g., the striatum or other ROI, injection of radiolabeled DaT2020 should be monitored starting at the injection to make sure it has entered the circulating system from its injection site. This can be accomplished by any method known in the art, such as, but not limited to, the LaraSystem (Lucerno Dynamics).

For example, should the administered radiolabeled DaT2020 cause an infiltration at the injection site or should it be limited to the site of a venous blockage, the label will not, or more slowly, get to the brain or ROI, or very little of it will get to the site of DATs, resulting in a false reading or no or low DAT, and potentially leading to a false diagnosis of a dopaminergic disorder.

### M. DaT2020 vs DaTscan

Dat2020 travels in the blood stream and into the brain more quickly and binds more specifically to DAT than DaTscan. Because of the favorable pharmacokinetic profile of DaT2020 stable binding occurs 15 min after administration allowing scanning to begin and resulting in sufficient data for an image to be collected over 30 min. In contrast, the scanning procedure with DaTscan cannot begin until at least 180 min after administration and usually the scan lasts about 45 min.

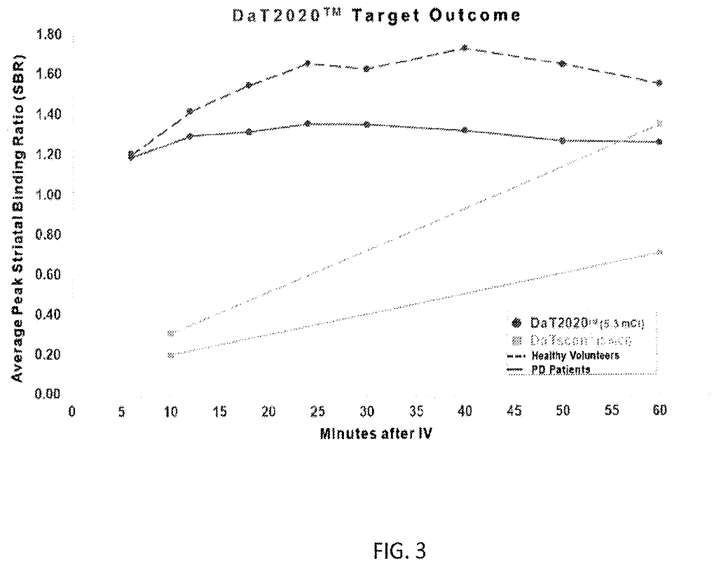

FIG. 3 displays results of SPECT data from which striatal uptake (striatal binding ratio (“SBR”)) of DaTscan or of radiolabeled DaT2020 was calculated at intervals over a 60-min period after IV injection of either of 8 mCi DaT2020 or 5.3 mCi DaTscan of healthy volunteers or mild Parkinson's patients (see EXAMPLE 2). The results obtained from administering lower doses of radiolabeled_DaT2020 (5.3 mCi) were modeled from the data obtained using the 8 mCi dose and a 3-headed camera. Within six min after injection of radiolabeled DaT2020, the specific binding ratios of Parkinson's patients and the healthy volunteer group had reached 90% and 74% of the average stable binding ratio of 1.32 and 1.64, respectively, during the previously determined imaging time of 15 min to 45 min post-administration. In contrast, DaTscan takes least 180 min to reach a stable binding ratio.

Thus, unlike DaTscan, radiolabeled DaT2020 rapidly binds to DAT and achieves stable binding 10 min to 15 min after injection with high selectivity (28-fold DAT:SERT), and progresses by diffusion quickly and deeply into the brain parenchyma after traversing the blood-brain-barrier.

Reference will now be made to specific examples illustrating the disclosure. It is to be understood that the examples are provided to illustrate exemplary embodiments and that no limitation to the scope of the disclosure is intended thereby.

### Example 1

### Diagnosis of a Movement Disorder

A patient exhibiting a movement disorder clinically diagnosed by a physician is positioned in a SPECT camera [with or without improved resolution capabilities (e.g., Discovery NM-630, GE Healthcare, Inc., Chicago, Ill. or inSPira HD®, Samsung Neurologica Corporation, Danvers, Mass.). Access into a large vein (e.g., antecubital vein) is established using an 18 gauge to 22 gauge indwelling polyurethane catheter that does not contain silicone (e.g., Bard® Poly Midline, C.R. Bard, Inc., Salt Lake City, Utah). To avoid extravasation of [¹²³I]-DaT2020, correct localization of the catheter is ensured by a test injection of normal saline prior to the injection of [¹²³I]-DaT2020. Then the patient is slow injected with 3.5±1.0 mCi (296 MBq) [¹²³I]-DaT2020 (LikeMinds, Boston, Mass.), followed by a 10 mL saline flush.

To determine the exact radioactive dose administered the difference between the radioactivity in the syringe and delivery system before and after injection is calculated. After the dose is delivered, the syringe is filled with a volume of saline equal to the administered dose volume. The syringe content is recounted under the same conditions as used to determine the dose separately. The delivery system is placed in a plastic container and counted in a dose calibrator (e.g., CRC®-25R Doe Calibrator, Capintec, Inc., Florham Park, N.J.) using the same parameters as used for the dose. Measured radioactivity values and times of measurement are documented in the source documents and recorded in the patient record, as well as the total injected volume. Injected radioactivity values outside the above stated range, i.e., values lower than about 3 mCi or higher than about 4 mCi are considered as potential sources of variation.

Acquisition is in “step and shoot” mode with each head rotating 360 degrees using a parallel hole collimator (GE Healthcare, Inc., Chicago, Ill.) used to create the tomograph to permit the possible reconstruction of a viable image (even if one head is faulty.)

Specific SPECT scan parameters, including collimation and acquisition mode, are set out below. Raw projection data is acquired into a 128×128 matrix, stepping each head 3 degrees for a total of 120 projections into a 20% symmetric photopeak window centered on 159 keV for a total scan duration of approximately 10 min. The strength of the photon emission is then calculated.

Signals acquired by the scanner initiated at 15 min after tracer administration are processed in a computer system running e software which is camera- and SPECT system-ignorant. If the patient's movement disorder is not a dopaminergic disorder, then within 10 to 15 min after initiating count acquisition, there will be a normal level of counts collected. If the number of counts is lower than normal, the movement disorder is likely a dopaminergic disorder. The patient can then be treated with medicaments known to be efficacious for the type of disorder diagnosed.

Within 30 min to 45 min after that, the computer/software assembly then assembles a dynamic image of gradual DaT2020 uptake into the ROI as indicated by received counts, and also provide static images at time points appropriate with the underlying pathology. For example, Hermes Medical Solutions (Grenenvielle, N.C.) offers Hybrid Recon™ (https://www.hermesmedical.com/products/suv-spect-reconstruction-absolute-quantification/) for use in reconstructing SPECT data, and producing quantitative output in the form of standardized uptake value. In the case of a dopaminergic disorder, the image of the striatum obtained will be asymmetrical and lighter; relative to the symmetrical and bright image obtained from a patient experiencing a non-dopaminergic movement disorder

### Example 2

### Comparison of DaT2020 and DaTscan as Imaging Agents

The following studies demonstrate that DaT2020 is a superior to DaTscan for determining if a tremor disorder manifested by a patient is a non-dopaminergic tremor disorder (e.g., essential tremor) or is a dopaminergic movement disorder (e.g., Parkinson's disease).

### A. DaT2020 Study

The subjects used in the study were healthy volunteers (HV) and those with at least mild Parkinson's disease (PD) (Seibyl at al. (2008) “ALTROPANE SPECT in Parkinson's disease patients and healthy controls” (poster). Abs. Movement Dis. Soc. 12th Internat. Con. Parkinson's Disease and Movement Disorders). All subjects were evaluated by a clinician using validated measures to determine if there is evidence of a movement disorder. Any subject using an anti-parkinsonian medication abstained from its use for at least 12 hr prior to the movement disorder evaluation. All subjects received a thyroid blocker and waited the specified time for it to take effect (1 h to 12 h, depending on blocker used) before intravenous injection (IV) of DaT2020. After thyroid blocking was achieved, subjects were positioned in the SPECT camera and administered a single bolus IV of 296 MBq (8 mCi) [range 37 to 296 MBq] of DaT20201.

The SPECT camera used had 3 heads, each of which rotates 360 degrees sampling every 3 degrees for a total of 120 raw projection images per head. Projection data was collected in a 128×128 matrix into a symmetric energy window centered at 159 kEv±10%. Using this acquisition protocol permits post hoc analysis of imaging data at each time point using information from 1, 2, or 3 more heads. This, in turn, enables modeling the impact of different doses of DaT2020; 2.7 mCi, 5.3 mCi, and 8 mCi, respectively

SPECT scans were acquired for a total of 60 min beginning immediately after administration of DaT2020. The first 5 scans were dynamic and lasted 6 min each for a total of 30 min. Immediately following the dynamic scans, 3 static scans (10 min each) were acquired for a total of 30 min.

Head movement within each of the 8 scan sessions was checked by using five external skin fiducial markers containing 1 μCi of ¹²³I placed along the canthomeatal line (2 right side, 3 left side) prior to each SPECT scan.

Data was re-constructed using filtered back projection and a simple ramp filter followed by a post hoc (3-D) standardized low pass filter. Attenuation correction was performed by applying a Chang 0 correction and a mu of 0.11 cm-1 using a validated automated software algorithm.

ROIs were determined using data obtained from about 24 min to 30 min after dose administration, with individual subject ROI sampling of the left and right caudate and putamen and an occipital background region. These ROIs were then applied to all images obtained during the scanning session (for a total of 24, assuming 8 time-points×3 camera head conditions: 1, 2, or all 3 heads).

To determine the striatal binding ratios (SBR) extract total counts within the ROI, total volume, and count density (counts/voxel) from each scan. SBR is calculated as the density of counts (counts per voxel per min) in the striatal region minus the counts in the occipital cortex (background) divided by the density of counts in the occipital background region. The mean striatal SBR scores are calculated as the mean of the left and right caudate and putamen SBR scores.

$$SBR = \frac{\begin{matrix}mean counts in region of interest - \\ counts in occipital cortex\end{matrix}}{counts in occipital cortex}$$

DaT2020 SBR data over time were abstracted by reading the graphics showing SBR over time for PD patients (Seibyl and Merck, ibid., Appendix 1) (the first scan of two was read), and in Appendix 2 for healthy volunteers (Seibyl and Merck, ibid., Appendix 2). The acquisition protocol permitted the post hoc analysis of imaging data at each time point using information from 1 or 2 heads, hence modeling the impact of different injected doses of ¹²³I DaT2020 at 2.7 mCi and 5.3 mCi, respectively.

### B. DaTscan Study

This study was performed as described in the FDA (2011) Clinical Pharmacology Review https://www.accessdata.fda.gov/drugsatfda_docs/nda/2011/022454sOrig1s000ClinPharmR.pdf.

All human adult subjects (ages 40 to 70) are evaluated by a clinician using validated measures to determine if there is evidence of a movement disorder. If the subject is currently using an antiparkinsonian medication, the subject must be willing and medically able to abstain from the medication for at least 12 hr prior to the evaluation. All subjects must receive a thyroid blocker and wait the specified time for it to take effect (1 hr to 12 hr, depending on blocker used) before IV of DaTscan.

The first scan is static and acquired beginning 10 min after administration of DaTscan. This scan involves multislice SPECT acquisition (starting at and parallel to the orbitomeatal line, 150 sec per slice; interslice distance 10 mm) to locate the slice that demonstrates the most useful visualization of the striatum. The remaining 5 scans (approximately 20 min each) are dynamic (8 consecutive acquisitions of 150 sec per slice) performed at the level of the reference slice determined during the static scan; and acquired at 1 h, 2 h, 3 h, 4.5 h, and 6 hr post-administration of DaTscan. Projection data is collected in a 128×128 matrix into a symmetric energy window set at 135 to 190 kEv.

Images are automatically reconstructed with a variable filter, according to the level of counts per slice. Linear attenuation correction is based on an absorption length of 95 mm.

To analyze striatal DaTscan binding for data obtained during static scanning, use a standard template with regions of interest for the whole striatum, caudate nucleus, putamen, and occipital cortex (OCC) positioned on the slice with the highest activity. Use the same template to analyze striatal DaTscan binding from images obtained during dynamic scanning. Small variations of individual brains will require movement of the fixed regions of interest, without changing the size and shape, within the template for optimal fitting. Specific to non-specific DaTscan binding is calculated as:

DaTscan binding=(ROI−OCC)/OCC

in which ROI represents the mean radioactivity in the region of interest (striatum, caudate nucleus or putamen).

### C. Comparison of Study Parameters

The following are summary tables of relevant factors for both studies.

TABLE 1
Study, Subjects, and Number and Timing of SPECT Scans

| | DaT2020 Seibyl & Marek | DaTscan CY96-FP2-CSR |
|---|---|---|
| Study type | Single center, open label study | Single center, open study |
| Dose | 296 MBq[sic] (8 mCi) | 111 MBq (3 mCi) |
| | NOTE: Using 2 heads | |
| | approximates 5.3 mCi. This is data | |
| | in schematic. | |
| Healthy Volunteers SSs (HV) | N = 12, 4 F, age range 55-77 | n = 10, age range 40-70 |
| Healthy confirmation | evaluated by a movement disorder | Not reported |
| | specialist to confirm the absence of | |
| | neurologic illness | |
| Parkinson's disease Patients | N = 15, 4 F, age range 45-85 | n = 20, age range 40-70 |
| (PD) | | |
| PD diagnosis | Hoehn & Yahr average 1.9 | H&Y 1 (hemi-PD) |
| | UPDRS exam by movement | |
| | disorder specialist following | |
| | overnight period without anti- | |
| | parkinson medication | |
| Total scan time post IV | 60 min | Total of approximately 120 min |
| | | (approximately 20 min for each of 6 |
| | | scans) over 6 hr |
| Static Scans | 0 | 1 |
| Timing--post IV Peak SBR | 0 | 10 min |
| (static) | | |
| Dynamic scans | 8 | 5 |
| Timing--post IV Peak SBR | Five 6-min scans | 1, 2, 3, 4.5 and 6 hr |
| (dynamic) | Followed by three 10-min scans | |
| | NOTE: specific times not given | |

TABLE 2
SPECT Camera, Image Acquisition, and Preparation

| | DaT2020 | DaTscan |
|---|---|---|
| Camera | Philips PRISM 3000XP triple-headed camera | Strichman Medical Equipment 810X |
| Heads | 3 | |
| Head rotation | 360 degrees | Not reported |
| Collimator | fan-beam collimators | 12 individual crystals, each equipped |
| | | with a focusing collimator |
| Energy window | 159 ± 10% kEv [sic] | 135-190 keV |
| Matrix | 128 × 128 | 128 × 128 |
| Optimum time of | Peak SBR assessed for each SS for each scan | time at which the specific |
| acquisition | | radioactivity in the striatum reached |
| | | its highest value in all subjects |
| Raw images | Sampling every 3 degrees. Total of 120 raw | Not reported |
| | images per head | |
| Image | using filtered back projection and a simple | automatically reconstructed using |
| reconstruction | ramp filter. followed by a post hoc (3-D) | variable filter, according to level of |
| | standardized low pass filter. | counts per slice |
| Attenuation | performed applying a Chang 0 correction and a | linear attenuation correction, based |
| correction | mu of 0.11 cm-1 using an automated MNI | on an absorption length of 95rnm |
| | software algorithm. | was applied in all studies [sic] |
| Region of Interest | Determined in the 3-head data from dynamic | In static scan, standard template for |
| (ROI) | scan #4 or #5 with individual ROI sampling of | whole striatum. Used same template |
| | left and right caudate and putamen and an | for dynamic scans with position (not |
| | occipital background region. This ROI was | size) adjustments for individual SS |
| | then applied to all images during the scan. | |

TABLE 3
Calculating Peak SBR

| | DaT2020 | DaTscan |
|---|---|---|
| Corrections to | | corrected for radioactive decay and |
| Individual S data | | averaged |
| SBR calculation | Total counts within the ROI, total volume, | Strichman Medical Units (SMU's; 1 |
| | and count density (counts/voxel) were | SMU = 100 Bq/mL) |
| | extracted from each scan and logged in a | (ROI − OCC)/OCC |
| | data spreadsheet for determination of | where ROI represents the mean |
| | striatal uptake ratios (SBR) defined as the | radioactivity (in SMU) in the region |
| | density of counts (counts per voxel per | of interest; occipital cortex (OCC, |
| | min) in the striatal region divided by the | radioactivity in SMU) |
| | density of counts in the occipital | |
| | background region. | |

### D. Results

The results are shown in FIG. 3 for healthy volunteers and Parkinson's PD subjects receiving 3 mCi DaTscan and healthy volunteers and Parkinson's patients receiving 5.3 mCi DaT2020 as modeled from the 8 mCi (3 head) study of healthy volunteers and Parkinson's patients.

### Example 3

### Diagnosis and Development of a Dopaminergic Disorder

A subject who does not clinically manifest a dopaminergic disorder, but who has a family history of the disorder, is scanned for the presence of radiolabeled DaT2020 bound to DAT in a ROI (e.g., the striatum, putamen, kidney, pancreas) to determine the propensity of the subject to develop the clinical manifestations at a later time.

The subject is administered radiolabeled DaT2020 IV, and then after about 15 min, and for at least 30 min, the subject is scanned for radioactivity in the ROI. The counts from radiolabeled DaT2020 is collected by SPECT, PET, or by any sensor reader capable of monitoring the radiolabel.

The counts and/or pattern and intensity of radiolabeled DaT2020 binding to DAT at the ROI in the subject is then compared with the average results obtained from normal (no clinical manifestations or family history of disease), age-matched subjects (controls) to detect any differences, and if so, if these differences are substantial and/or credible. For example, the number, location, and/or pattern of DAT binding can be indicative of the presence or absence of the disorder.

The same procedure is repeated at intervals of e.g., 1 to 5 years to determine if and how the disorder starts to develop (if not difference from control was detected prior to the subsequent screenings), and then how it progresses with time and potentially, after treatment.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of determining if a subject not manifesting a clinical symptom of a dopaminergic disorder is afflicted with the dopaminergic disorder, comprising:
administering radiolabeled DaT2020, or a radiolabeled derivative thereof, to the subject; and
acquiring counts from the radiolabeled DaT2020, or derivative thereof, bound to DAT in a region of interest (ROI) of the body of the subject, initiation of the acquisition of counts beginning at about 15 min after administration;
measuring a number, density, and/or pattern of counts acquired; and
comparing the number, density, and/or pattern of counts acquired from the ROI of the subject with the number, density, and/or pattern of counts obtained from an unafflicted, age-matched control subject,
the patient being afflicted with a dopaminergic movement disorder if the number, density and/or pattern of counts detected in the ROI is reduced relative to the counts, density, and/or pattern of counts obtained from the ROI the unafflicted, age-match control subject.

2. The method of claim 1, further comprising repeating the method at a set period of time after the method is first performed.

3. The method of claim 1, wherein the counts obtained from the unafflicted, age-matched control subject is an average of counts, density, and/or patterns obtained from a plurality of unafflicted, age-matched control subjects.

4. The method of claim 1, wherein the ROI is the striatum, putamen, kidney, pancreas, or a part of the cardio-vascular system of the subject.

5. The method of claim 1, wherein DaT2020, or a derivative thereof, is radiolabeled with ¹²³I, ¹²⁴I, ¹²⁵I, ^{99m}Tc, ¹⁸F or ^{117m}Sn.

6. The method of claim 1, wherein DaT2020, or a derivative thereof, is radiolabeled with ¹²³I, ¹²⁵I, ^{99m}Tc, or ^{117m}Sn, and counts are acquired by SPECT.

7. The method of claim 1, wherein DaT2020, or a derivative thereof, is radiolabeled with ¹⁸F, ¹²⁴I, or ¹¹C, and the counts are acquired by PET.

8. The method of claim 1, wherein about 1 mCi to about 10 mCi ¹²³I-labeled DaT2020, or a derivative thereof, is administered to the subject.

9. The method of claim 8, wherein about 3 mCi to about 5 mCi ¹²³I-labeled DaT2020, or a derivative thereof, is administered to the subject.

10. The method of claim 1, wherein the derivatives of DaT2020 comprise 2β-carbomethoxy-3β-(4-iodophenyl)tropane beta-CIT); 2β-carbomethoxy-3β-(4-iodophenyl)-N-(3-fluoropropyl)nortropane (FP-CIT) and TRODAT-1.

11. The method of claim 4, wherein the ROI is the striatum and the subject not manifesting a clinical symptom is afflicted with Parkinson's disease, Lewy Body dementia, or diabetes.

12. A method of determining if a patient manifesting active tremor symptoms is afflicted with a non-dopaminergic movement disorder or a dopaminergic movement disorder, the method comprising:
administering radiolabeled DaT2020, or a radiolabeled derivative thereof, to the patient;
acquiring counts from the radiolabeled DaT2020, or derivative thereof, bound to DAT in the striatum of the patient, initiation of the acquisition of counts beginning at about 15 min after administration;
measuring a number, density, and/or pattern of counts acquired; and
comparing the number, density, and/or pattern of counts acquired from the striatum of the patient with the number, density, and/or pattern of counts obtained from an unafflicted, age-matched control subject that does not exhibit active tremor symptoms,
the patient being afflicted with a dopaminergic movement disorder if the number, density and/or pattern of counts detected in the striatum of the patient is reduced relative to the counts, density, and/or pattern of counts obtained from the striatum of the unafflicted, age-match control subject, and
the patient. being afflicted with a non-dopaminergic movement disorder if the number, density and/or pattern of counts detected in the striatum of the patient is not reduced relative to the counts, density, and/or pattern of counts obtained from the striatum of the unafflicted, age-match control subject.

13. The method of claim 12, wherein the non-dopaminergic disorder afflicting the patient manifesting active tremor symptoms is essential tremor.

14. The method of claim 12 wherein the dopaminergic disorder afflicting the patient manifesting active tremor symptoms is Parkinson's disease, or Lewy Body dementia.
